(19) United States
(12) Patent Application Publication
(10) Pub. No.: US 2014/0274901 A1
(43) Pub. Date: Sep. 18, 2014

(54) PROANP COMPOSITIONS AND METHODS FOR TREATING ACUTE HEART FAILURE

(71) Applicant: Mayo Foundation for Medical Education and Research, Rochester, MN (US)

(72) Inventors: Tomoko Ichiki, Rochester, MN (US); John C. Burnett, JR., Rochester, MN (US)

(73) Assignee: Mayo Foundation for Medical Education and Research, Rochester, MN (US)

(21) Appl. No.: 14/205,700

(22) Filed: Mar. 12, 2014

Related U.S. Application Data
(60) Provisional application No. 61/776,891, filed on Mar. 12, 2013.

(51) Int. Cl. C07K 14/58

(52) U.S. Cl. CPC C07K 14/58
USPC 514/12.4

(57) ABSTRACT

Materials and Methods related to using proANP to treat cardiorenal disease, including acute heart failure.

### CROSS-REFERENCE TO RELATED APPLICATIONS

This application claims benefit of priority from U.S. Provisional Application Ser. No. 61/776,891, filed on Mar. 12, 2013.

### STATEMENT AS TO FEDERALLY SPONSORED RESEARCH

This invention was made with government support under HL036634 awarded by the National Institutes of Health. The government has certain rights in the invention.

### TECHNICAL FIELD

This document relates to proANP peptides, and methods for using proANP peptides to treat acute heart failure.

### BACKGROUND

Natriuretic polypeptides are polypeptides that can cause natriuresis—increased sodium excretion in the urine. Such polypeptides can be produced by brain, heart, kidney, and/or vascular tissue. The natriuretic polypeptide family in humans includes the cardiac hormones atrial natriuretic peptide (ANP), B-type natriuretic peptide (BNP), C-type natriuretic peptide (CNP), and urodilatin (URO). Natriuretic polypeptides function via well-characterized guanylyl cyclase receptors (i.e., NPR-A for ANP, BNP, and URO; and NPR-B for CNP) and the second messenger cyclic 3′5′ guanosine monophosphate (cGMP) (Kuhn (2003) Circ. Res. 93:700-709; Tawaragi et al. (1991) Biochem. Biophys. Res. Commun. 175:645-651; and Komatsu et al. (1991) Endocrinol. 129:1104-1106).

Cardiorenal syndrome (CRS) is characterized by both kidney failure and heart failure. The primarily failing organ can either be the heart or the kidney, and the failing organ often precipitates failure of the other. In some cases, for example, acute kidney injury or chronic kidney disease can lead to cardiac dysfunction (e.g., arrhythmia or heart failure (HF)). In other cases, chronic heart failure can lead to chronic kidney disease, and acute decompensated heart failure (ADHF) can lead to acute kidney injury.

ADHF is a worsening of the symptoms of heart disease, and is a common and potentially serious cause of acute respiratory distress. Patients with ADHF may experience shortness of breath, edema, and fatigue. Decompensation of chronic stable heart failure can result from an intercurrent illness such as pneumonia, myocardial infarction (MI), arrhythmias, uncontrolled hypertension, or a patient's failure to maintain a fluid restriction, diet, or medication. Other contributing factors include anemia, hyperthyroidism, excessive fluid or salt intake, and medication that causes fluid retention. See, e.g., Allen and O'Connor, Can Med Assoc J, 2007, 176(6):797-805; Fonarow et al., Arch Intern Med, 2008, 168(8):847-854; and Nieminen et al., Eur Heart J, 2005, 26(4):384-416.

### SUMMARY

This document is based in part on the discoveries that exogenous human proANP is processed to biologically active ANP in serum from normal subjects and HF patients, and that the processed mature ANP can stimulate human GC-A overexpressed in HEK293 cells. This document also is based in part on the observation that bolus proANP injection into normal canines resulted in a prolonged increase in both plasma ANP and cGMP levels, as well as a prolonged increase in urine volume, natriuresis, and cGMP excretion with minimal hemodynamic changes as compared to ANP injection. Further, in pharmacokinetic analyses, proANP had a half-life that was about 4 times longer than ANP. ProANP also may have anti-renin-angiotensin-aldosterone system (RAAS) effects, since it is processed to ANP and URO in the kidney, and thus it may be more efficacious than other therapeutics such as furosemide. Further, proANP is native to humans, such that its therapeutic use may lack untoward side effects, unlike small molecules. Thus, proANP may be useful as a therapeutic for CRS and its components. For example, proANP may be useful as a renal-specific drug for treating ADHF, without hypotension and with a longer half-life as compared to ANP.

In one aspect, this document features a method for treating a cardiorenal disease in a mammal in need thereof. The method can include administering to the mammal a proANP polypeptide in an amount effective to reduce a symptom of the cardiorenal disease. The cardiorenal disease can comprise acute decompensated heart failure. The proANP polypeptide can contain amino acids 21-126 of SEQ ID NO:3. The proANP polypeptide can contain SEQ ID NO:3, or can consist of SEQ ID NO:3. The method can include administering to the mammal a composition comprising the proANP polypeptide. The mammal can be a human. The proANP polypeptide can be administered at a dose of 0.01 ng/kg to 50 ug/kg. The method can include administering the proANP intravenously. The symptom can be selected from the group consisting of as edema, shortness of breath, and fatigue, cardiac unloading, increased glomerular filtration rate, decreased plasma renin activity, decreased levels of angiotensin II, decreased proliferation of cardiac fibroblasts, decreased left ventricular (LV) hypertrophy, decreased LV mass, decreased pulmonary wedge capillary pressure, decreased right atrial pressure, decreased mean arterial pressure, decreased levels of aldosterone, decreased ventricular fibrosis, increased ejection fraction, and decreased LV end systolic diameter. The method can further include identifying the mammal as being in need of the treatment.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram showing the sequences of human and canine preproANP (SEQ ID NOS:1 and 2, respectively). The dashed box indicates the signal peptide sequence. Bold type indicates amino acids that differ in the canine sequence as compared to the human sequence. The start of the 126 amino acid proANP sequence is indicated by the arrow. Italicized type indicates urodilatin sequences, and underlining indicates mature ANP sequences.

FIGS. 2A and 2B are diagrams depicting endogenous production, secretion, and processing of molecular forms of ANP in the heart and kidneys. ProANP (H), heart endogenous proANP; ProANP (K), kidney endogenous proANP. ProANP 1-126 (SEQ ID NO:3) is processed into ANP 1-28 (SEQ ID NO:4; also referred to as ANP) and NT-proANP 1-98 (SEQ ID NO:5). ProANP also is thought to be processed into urodilatin (URO; SEQ ID NO:6) and NT-proANP 1-94 (SEQ ID NO:7).

FIG. 3 is a diagram depicting processing of exogenous proANP [proANP (E)].

FIGS. 4A-4C show the results of an ex vivo study using proANP in normal and HF human serum. FIG. 4A is a schematic of His-tagged proANP1-126 processing. Exogenous proANP was incubated in human serum at 37° C. Processed and unprocessed proANP peptides were isolated by immunoprecipitation using His-tag beads, followed by detection via Western blotting with 6×His antibody. FIG. 4B shows a representative Western blot for proANP that was incubated in normal serum for 0 to 180 minutes, as indicated. FIG. 4C is a graph plotting densitometric analysis of processed ANP1-28 from proANP that was incubated in serum from normal subjects and HF patients for the indicated times. Values are mean±SEM. *p<0.05 vs. 0 min. There was no significant difference between the Normal and HF groups (2-way ANOVA).

FIGS. 5A-5D depict proANP effects in vitro. FIG. 5A is a pair of graphs plotting cGMP production in GC-A or GC-B overexpressing HEK293 cells treated with proANP, proBNP, or mature ANP or BNP, as indicated. *p<0.05 vs. non-treated cells. FIG. 5B is a pair of graphs plotting cGMP production in GC-A or GC-B overexpressing HEK293 cells treated with proANP (10⁻⁸ M) in serum or PBS, as indicated. *p<0.05 vs. non-treated cells. †p<0.05 vs. PBS-treated proANP. FIG. 5C is a series of photographs showing immunocytochemistry for collagen type I (Col I), GC-A, and corin in cardiac fibroblasts (CFs). NHS served as a negative control. Magnification is 100×. FIG. 5D is a graph plotting the time course of cGMP production in CFs treated with ANP or proANP (10⁻⁷M). Both ANP and proANP resulted in significant increases in cGMP levels at all time points as compared to 0 minutes. *p<0.05 vs. proANP at the same time point. Data are mean±SEM.

FIGS. 6A-6H are a series of graphs plotting proANP effects in vivo. Normal canines received one bolus injection of an equimolar dose (667 pmol/kg) of ANP (n=2), BNP (n=1) or proANP (n=2), and data were collected for 2 hours. FIG. 6A, plasma ANP and BNP levels; FIG. 6B, plasma cGMP levels; FIG. 6C, delta mean atrial pressure (MAP); FIG. 6D, heart rate (HR); FIG. 6E, cardiac output (CO); FIG. 6F, urine volume (UV); FIG. 6G, urinary sodium excretion (UNaV); FIG. 6H, renal blood flow (RBF). Data are mean±SEM.

FIG. 7 is a pair of graphs plotting area under the curve (AUC) for plasma (left panel) and urinary (right panel) cGMP after treatment of canines with proANP, ANP, or BNP as indicated.

### DETAILED DESCRIPTION

As described herein, exogenous human proANP is processed to biologically active ANP in serum from normal subjects and HF patients. Further, proANP injection into normal canines had prolonged natriuretic and diuretic effects, with minimal hemodynamic changes as compared to ANP. Thus, proANP may be useful as a therapeutic for ADHF.

### Natriuretic Polypeptides

Natriuretic polypeptides can be effective to increase plasma cGMP levels, increase urinary cGMP excretion, increase net renal cGMP generation, increase urine flow, increase urinary sodium excretion, increase urinary potassium excretion, increase hematocrit, increase plasma BNP immunoreactivity, increase renal blood flow, increase plasma ANP immunoreactivity, decrease renal vascular resistance, decrease proximal and distal fractional reabsorption of sodium, decrease mean arterial pressure, decrease pulmonary capillary wedge pressure, decrease right atrial pressure, decrease pulmonary arterial pressure, decrease plasma renin activity, decrease plasma angiotensin II levels, decrease plasma aldosterone levels, decrease renal perfusion pressure, and/or decrease systemic vascular resistance.

Amino acid sequences for endogenous human mature natriuretic polypeptides include the following:

| (SEQ ID NO: 4) | |
|---|---|
| ANP: | SLRRSSCFGGRMDRIGAQSGLGCNSFRY |
| (SEQ ID NO: 9) | |
| BNP: | SPKMVQGSGCFGRKMDRISSSSGLGCKVLRRH |
| (SEQ ID NO: 10) | |
| CNP: | GLSKGCFGLKLDRIGSMSGLGC |
| (SEQ ID NO: 6) | |
| URO: | TAPRSLRRSSCFGGRMDRIGAQSGLGCNSFRY |

The native Dendroaspis amino acid sequence for DNP is

(SEQ ID NO: 11)
EVKYDPCFGHKIDRINHVSNLGCPSLRDPRPNAPSTSA.

Each of these native mature natriuretic polypeptides includes a 17-amino acid ring structure with a cysteine bond between the cysteine residues at positions 1 and 17 (underlined in the above sequences) of the ring.

Natriuretic polypeptides include native (naturally occurring, wild type) natriuretic polypeptides such as ANP, BNP, CNP, and URO, as well as Dendroaspis natriuretic peptide (DNP)), pro and prepro forms of native natriuretic polypeptides, portions of native natriuretic polypeptides or pro or prepro forms of native natriuretic polypeptides, and variants of native natriuretic polypeptides or pro or prepro forms of native natriuretic polypeptides.

An “isolated” polypeptide is a polypeptide that (1) is not associated with proteins found in nature, (2) is free of other proteins from the same source (e.g., free of human proteins), (3) is expressed by a cell from a different species, or (4) does not occur in nature. An isolated polypeptide can be, for example, encoded by DNA or RNA, including synthetic DNA or RNA, or some combination thereof.

Human preproANP is a 151 amino acid polypeptide produced from the NPPA gene. The canine preproANP is slightly shorter; representative human and canine preproANP sequences are shown in FIG. 1 (SEQ ID NOS:1 and 2, respectively). Cleavage of the signal peptide results in proANP, which is 126 amino acids in length in both human (SEQ ID NO:3) and dog (SEQ ID NO:8). ProANP is stored in secretory granules in atrial cardiomyocytes, and upon secretion, proANP is processed into ANP 1-28 (SEQ ID NO:4; MW=3.08 KD) and amino-terminal (NT)-proANP 1-98 (SEQ ID NO:5) by the cardiac serine protease, corin (FIGS. 2A and 2B; Yan et al., Proc Natl Acad Sci USA, 2000, 97:8525-8529; and Yan et al., J Biol Chem, 1999, 274:14926-14935).

Alternative processing of filtered proANP in the kidney also is thought to generate NT-proANP 1-94 and urodilatin (URO; SEQ ID NO:6), which consists of the amino acid sequence of ANP with four additional amino acids at the N-terminus (MW=3.50 KD). URO is found in human urine, but has not been observed in human circulation or heart, suggesting that URO is produced and processed in the renal tubules and is cleaved by a different post translational process than ANP 1-28 (Potter et al., Endocr Rev, 2006, 27:47-72; Schulz-Knappe et al., Klin Wochenschr, 1988, 66:752-759; and Forssmann et al., Cardiovascular Res, 2001, 51:450-462).

The 28 amino acid human ANP peptide is released from the myocardium in response to various physiologic and pathophysiologic stimuli, such as myocardial wall stretch (FIG. 3). As described herein, proANP also may be released into the circulation intact, and then be processed to mature forms in the plasma and in other organ systems. These mature forms, ANP 1-28 and URO, are then degraded by neutral endopeptidase (NEP) 24.11, which is a membrane-bound metallopeptidase that has zinc at its active site and cleaves endogenous peptides to the amino side of hydrophilic residues. NEP is involved in the degradation of NPs, and is distributed in the kidney, lung, and vascular wall, but its highest concentration is in the kidney (Kenny and Stephenson, FEBS Letters, 1998, 232:1-8; and Kenny et al., Biochem J, 1993, 291(Pt 1):83-88). Insulin degrading enzyme (IDE) also is highly expressed in the kidney, and may be another enzyme that degrades ANP/URO (Ralat et al., J Biol Chem, 2011, 286:4670-4679).

Natriuretic polypeptides having one or more amino acid substitutions relative to a native natriuretic polypeptide amino acid sequence (also referred to herein as “variant” natriuretic polypeptides) can be prepared and modified as described herein. Amino acid substitutions can be made, in some cases, by selecting substitutions that do not differ significantly in their effect on maintaining (a) the structure of the peptide backbone in the area of the substitution, (b) the charge or hydrophobicity of the molecule at the target site, or (c) the bulk of the side chain. For example, naturally occurring residues can be divided into groups based on side-chain properties: (1) hydrophobic amino acids (norleucine, methionine, alanine, valine, leucine, and isoleucine); (2) neutral hydrophilic amino acids (cysteine, serine, and threonine); (3) acidic amino acids (aspartic acid and glutamic acid); (4) basic amino acids (asparagine, glutamine, histidine, lysine, and arginine); (5) amino acids that influence chain orientation (glycine and proline); and (6) aromatic amino acids (tryptophan, tyrosine, and phenylalanine) Substitutions made within these groups can be considered conservative substitutions. Non-limiting examples of useful substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenyalanine, glycine for proline, threonine for serine, serine for threonine, tyrosine for tryptophan, phenylalanine for tyrosine, and/or leucine for valine.

Any amino acid residue set forth in SEQ ID NO:3 can be subtracted, and any amino acid residue (e.g., any of the 20 conventional amino acid residues or any other type of amino acid such as ornithine or citrulline) can be added to the sequence set forth in SEQ ID NO:3. In some cases, a polypeptide provided herein can contain chemical structures such as ε-aminohexanoic acid; hydroxylated amino acids such as 3-hydroxyproline, 4-hydroxyproline, (5R)-5-hydroxy-L-lysine, allo-hydroxylysine, and 5-hydroxy-L-norvaline; or glycosylated amino acids such as amino acids containing monosaccharides (e.g., D-glucose, D-galactose, D-mannose, D-glucosamine, and D-galactosamine) or combinations of monosaccharides.

Further examples of conservative substitutions that can be made at any position within the polypeptides provided herein are set forth in Table 1.

In some embodiments, a natriuretic polypeptide can include one or more non-conservative substitutions. Non-conservative substitutions typically entail exchanging a member of one of the classes described above for a member of another class. Such production can be desirable to provide large quantities or alternative embodiments of such compounds. Whether an amino acid change results in a functional polypeptide can readily be determined by assaying the specific activity of the peptide variant using, for example, methods disclosed herein.

TABLE 1
Examples of conservative amino acid substitutions

| Original | | Preferred |
|---|---|---|
| Residue | Exemplary substitutions | substitutions |
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro | Pro |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn | Arg |
| Met | Leu, Phe, Ile | Leu |
| Phe | Leu, Val, Ile, Ala | Leu |
| Pro | Gly | Gly |
| Ser | Thr | Thr |
| Thr | Ser | Ser |
| Trp | Tyr | Tyr |
| Tyr | Trp, Phe, Thr, Ser | Phe |
| Val | Ile, Leu, Met, Phe, Ala, Norleucine | Leu |

In some embodiments, a polypeptide as provided herein can have a length of, for example, between 100 and 150 amino acid residues (e.g., between 105 and 145, between 110 and 140, between 115 and 135, between 120 and 131, between 121 and 130, between 122 and 129, between 123 and 128, between 124 and 128, or between 125 and 127 amino acid residues). It will be appreciated that a polypeptide with a length of 100 or 150 amino acid residues is a polypeptide with a length between 100 and 150 amino acid residues. In some embodiments, a natriuretic polypeptide can be an N-terminal truncation of SEQ ID NO:3. For example, a natriuretic polypeptide can include amino acids 5-126, 11-126, 15-126, 21-126, or 26-126 of SEQ ID NO:3.

Natriuretic polypeptides provided herein typically are cyclic due to disulfide bonds between cysteine residues. In some embodiments, a sulfhydryl group on a cysteine residue can be replaced with an alternative group (e.g., —CH₂CH₂—). To replace a sulfhydryl group with a —CH₂— group, for example, a cysteine residue can be replaced by alpha-aminobutyric acid. Such cyclic analog polypeptides can be generated, for example, in accordance with the methodology of Lebl and Hruby ((1984) Tetrahedron Lett. 25:2067), or by employing the procedure disclosed in U.S. Pat. No. 4,161,521.

In addition, ester or amide bridges can be formed by reacting the OH of serine or threonine with the carboxyl group of aspartic acid or glutamic acid to yield a bridge having the structure —CH₂CO₂CH₂—. Similarly, an amide can be obtained by reacting the side chain of lysine with aspartic acid or glutamic acid to yield a bridge having the structure —CH₂C(O)NH(CH)₄—. Methods for synthesis of these bridges are known in the art (see, e.g., Schiller et al. (1985) Biochem. Biophys. Res. Comm. 127:558, and Schiller et al. (1985) Int. J. Peptide Protein Res. 25:171). Other bridge-forming amino acid residues and reactions are provided in, for example, U.S. Pat. No. 4,935,492. Preparation of peptide analogs that include non-peptidyl bonds to link amino acid residues also are known in the art. See, e.g., Spatola et al. (1986) Life Sci. 38:1243; Spatola (1983) Vega Data 1(3); Morley (1980) Trends Pharm. Sci. 463-468; Hudson et al. (1979) Int. J. Pept. Prot. Res. 14:177; Spatola, in Chemistry and Biochemistry of Amino Acid Peptides and Proteins, B. Weinstein, ed., Marcel Dekker, New York, p. 267 (1983); Hann (1982) J. Chem. Soc. Perkin Trans. 1:307; Almquist et al. (1980) J. Med. Chem. 23:1392; Jennings-White et al. (1982) Tetrahedron Lett. 23:2533; European Patent Application EP 45665; Holladay et al. (1983) Tetrahedron Lett. 24:4401; and Hruby (1982) Life Sci. 31:189.

In some embodiments, a natriuretic polypeptide can comprise an amino acid sequence as set forth in SEQ ID NO:3, but with a particular number of amino acid substitutions. For example, a natriuretic polypeptide can have the amino acid sequence of SEQ ID NO:3, but with one, two, three, four, or five amino acid substitutions. Examples of such amino acid sequences include, without limitation, proANP with two arginine residues added to the C-terminus of the peptide, substitution of the valine at position 9 of proANP with a leucine, alanine, or lysine, substitution of the Gly-Arg-Met residues at positions 133-135 of proANP with Leu-Lys-Leu or Arg-Lys-Met, and substitution of the Gly-Ala-Gln residues at positions 139-141 of proANP with Gly-Ser-Met or Ser-Ser-Ser.

In some embodiments, a natriuretic polypeptide as provided herein can include an amino acid sequence with at least 85% (e.g., 85%, 86%, 87%, 88%, 89%, 90%, 91%, 92%, 93%, 94%, 95%, 96%, 97%, 97.5%, 98%, 98.5%, 99.0%, 99.5%, 99.6%, 99.7%, 99.8%, 99.9%, or 100%) sequence identity to a region of a reference natriuretic polypeptide sequence (e.g., SEQ ID NO:3). Percent sequence identity is calculated by determining the number of matched positions in aligned amino acid sequences, dividing the number of matched positions by the total number of aligned amino acids, and multiplying by 100. A matched position refers to a position in which identical amino acids occur at the same position in aligned amino acid sequences. Percent sequence identity also can be determined for any nucleic acid sequence.

The percent sequence identity between a particular nucleic acid or amino acid sequence and a sequence referenced by a particular sequence identification number is determined as follows. First, a nucleic acid or amino acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained online at fr.com/blast or at ncbi.nlm.nih.gov. Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to -1; -r is set to 2; and all other options are left at their default setting. For example, the following command can be used to generate an output file containing a comparison between two sequences: C:\Bl2seq c:\seq1.txt -c:\seq2.txt -p blastn -o c:\output.txt -q -1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence (e.g., SEQ ID NO:3), or by an articulated length (e.g., 100 consecutive nucleotides or amino acid residues from a sequence set forth in an identified sequence), followed by multiplying the resulting value by 100. For example, an amino acid sequence that has 120 matches when aligned with the sequence set forth in SEQ ID NO:3 is 95.2 percent identical to the sequence set forth in SEQ ID NO:1 (i.e., 120÷126×100=95.2). It is noted that the percent sequence identity value is rounded to the nearest tenth. For example, 75.11, 75.12, 75.13, and 75.14 is rounded down to 75.1, while 75.15, 75.16, 75.17, 75.18, and 75.19 is rounded up to 75.2. It also is noted that the length value will always be an integer.

Isolated polypeptides can be produced using any suitable methods, including solid phase synthesis, and can be generated using manual techniques or automated techniques (e.g., using an Applied BioSystems (Foster City, Calif.) Peptide Synthesizer or a Biosearch Inc. (San Rafael, Calif.) automatic peptide synthesizer. Disulfide bonds between cysteine residues can be introduced by mild oxidation of the linear polypeptides using KCN as taught, e.g., in U.S. Pat. No. 4,757,048. Natriuretic polypeptides also can be produced recombinantly, as described herein.

In some cases, a polypeptide provided herein can be a substantially pure polypeptide. As used herein, the term “substantially pure” with reference to a polypeptide means that the polypeptide is substantially free of other polypeptides, lipids, carbohydrates, and nucleic acid with which it is naturally associated. Thus, a substantially pure polypeptide is any polypeptide that is removed from its natural environment and is at least 60 percent pure or is any chemically synthesized polypeptide. A substantially pure polypeptide can be at least about 60, 65, 70, 75, 80, 85, 90, 95, or 99 percent pure. Typically, a substantially pure polypeptide will yield a single major band on a non-reducing polyacrylamide gel.

Salts of carboxyl groups of polypeptides can be prepared by contacting the peptide with one or more equivalents of a desired base such as, for example, a metallic hydroxide base (e.g., sodium hydroxide), a metal carbonate or bicarbonate base (e.g., sodium carbonate or sodium bicarbonate), or an amine base (e.g., triethylamine, triethanolamine, and the like). Acid addition salts of polypeptides can be prepared by contacting the polypeptide with one or more equivalents of an inorganic or organic acid (e.g., hydrochloric acid).

Esters of carboxyl groups of polypeptides can be prepared using any suitable means (e.g., those known in the art) for converting a carboxylic acid or precursor to an ester. For example, one method for preparing esters of the present polypeptides, when using the Merrifield synthesis technique, is to cleave the completed polypeptide from the resin in the presence of the desired alcohol under either basic or acidic conditions, depending upon the resin. The C-terminal end of the polypeptide then can be directly esterified when freed from the resin, without isolation of the free acid.

Amides of polypeptides can be prepared using techniques (e.g., those known in the art) for converting a carboxylic acid group or precursor to an amide. One method for amide formation at the C-terminal carboxyl group includes cleaving the polypeptide from a solid support with an appropriate amine, or cleaving in the presence of an alcohol, yielding an ester, followed by aminolysis with the desired amine.

N-acyl derivatives of an amino group of a polypeptide can be prepared by utilizing an N-acyl protected amino acid for the final condensation, or by acylating a protected or unprotected peptide. O-acyl derivatives can be prepared for example, by acylation of a free hydroxy peptide or peptide resin. Either acylation may be carried out using standard acylating reagents such as acyl halides, anhydrides, acyl imidazoles, and the like. Both N- and O-acylation may be carried out together, if desired.

In some embodiments, the natriuretic polypeptides provided herein can have half-lives that are increased relative to the half-lives of other natriuretic polypeptides. For example, while the half-life of ANP in humans is about two to five minutes and its metabolic clearance rate is about 14 to 25 ml/min/kg, the elimination half-life of a proANP peptide (e.g., containing SEQ ID NO:3) can be increased by comparison. In some cases, a natriuretic polypeptide provided herein can have a half-life that is increased by at least 2-fold (e.g., at least 2-fold, at least 3-fold, at least 4-fold, at least 5-fold, at least 6-fold, at least 7-fold, at least 8-fold, at least 9-fold, or at least 10-fold) as compared to a native natriuretic polypeptide such as ANP, for example. In some embodiments, a natriuretic polypeptide can have an elimination half-life of at least about 5 minutes (e.g., at least about 5 minutes, at least about 7 minutes, at least about 10 minutes, at least about 12 minutes, at least about 15 minutes, at least about 17 minutes, at least about 18 minutes, or at least about 20 minutes).

In some embodiments, a natriuretic polypeptide can be modified by linkage to a polymer such as polyethylene glycol (PEG), or by fusion to another polypeptide such as albumin, for example. In some embodiments, one or more PEG moieties can be conjugated to a natriuretic polypeptide via lysine residues. Linkage to PEG or another suitable polymer, or fusion to albumin or another suitable polypeptide can result in a modified natriuretic polypeptide having an increased half-life as compared to an unmodified natriuretic polypeptide. Without being bound by a particular mechanism, an increased serum half-life can result from reduced proteolytic degradation, immune recognition, or cell scavenging of the modified natriuretic polypeptide. Methods for modifying a polypeptide by linkage to PEG (also referred to as “PEGylation”) or other polymers are known in the art, and include those set forth in U.S. Pat. No. 6,884,780; Cataliotti et al. ((2007) Trends Cardiovasc. Med. 17:10-14; Veronese and Mero (2008) BioDrugs 22:315-329; Miller et al. (2006) Bioconjugate Chem. 17:267-274; and Veronese and Pasut (2005) Drug Discov. Today 10:1451-1458, all of which are incorporated herein by reference in their entirety. Methods for modifying a polypeptide by fusion to albumin also are known in the art, and include those set forth in U.S. Patent Publication No. 20040086976, and Wang et al. (2004) Pharm. Res. 21:2105-2111, both of which are incorporated herein by reference in their entirety.

A natriuretic polypeptide as provided herein can function through one or more of the guanylyl cyclase receptors through which the native natriuretic polypeptides function. For example, a natriuretic polypeptide as provided herein can bind to and function through the NPR-A receptor through which ANP and BNP function, or through the NPR-B receptor through which CNP functions. Further, in some cases, a natriuretic polypeptide as provided herein can bind to and function through more than one guanylyl cyclase receptor, including NPR-A and NPR-B, for example. Methods for evaluating which receptor is involved in function of a particular natriuretic polypeptide are known in the art. For example, glomeruli, which contain both NPR-A and NPR-B, can be isolated (e.g., from a laboratory animal such as a dog) and incubated with a natriuretic polypeptide (e.g., a native or mutated natriuretic polypeptide), and cGMP levels can be measured. Glomeruli can be pretreated with antagonists of NPR-A or NPR-B to determine whether cGMP production stimulated by a natriuretic polypeptide through one or the other receptor can be attenuated.

In some cases, an isolated natriuretic polypeptide and herein can be used to treat cardiorenal disease. For example, an isolated natriuretic polypeptide (e.g., proANP) can be used to treat acute HF (e.g., ADHF). The presence or extent of cardiorenal disease can be evaluated using methods known in the art, including, without limitation, general clinical examination to evaluate blood pressure, heart rate, heart rhythm, arterial oxygen, and hemoglobin levels; echocardiography to measure ejection fraction, LV and left atrium (LA) diameter, LV wall motion, LV filling pressure, and diastolic function by pulse and tissue Doppler; use of a Swan-Ganz catheter to measure cardiac output, pulmonary wedge pressure, pulmonary arterial pressure, right ventricle pressure, right atrium pressure, and systemic and pulmonary vascular resistance; assessment of kidney function by determination of glomerular filtration rate, serum creatinine, and blood urea nitrogen; and measurement of biomarkers such as BNP, amino-terminal proBNP (NT-proBNP), troponin-T, troponin-I, C-reactive protein (CRP), and creatin-kinase, serum cystatin-C, albuminuria, neutrophil gelatinize associated lopocalin (NGAL), N-acetyl-beta-D-glucosaminidase (NAG), kidney injury molecule-1 (KIM-1), angiotensin-II, renin, aldosterone, and inflammatory cytokines (e.g., interleukin (IL)-6, IL-18, etc.). In some cases, an isolated natriuretic polypeptide as provided herein can reduce one or more symptoms of acute HF, including clinical parameters such as edema, shortness of breath, and fatigue, as well as cardiac unloading (i.e., reduced pressure in the heart), increased glomerular filtration rate (GFR), decreased plasma renin activity (PRA), decreased levels of angiotensin II, decreased proliferation of cardiac fibroblasts, decreased left ventricular (LV) hypertrophy, decreased LV mass (indicative of reduced fibrosis and hypertrophy), decreased pulmonary wedge capillary pressure (PWCP; an indirect measure of left atrial pressure), decreased right atrial pressure, decreased mean arterial pressure, decreased levels of aldosterone (indicative of an anti-fibrotic effect), decreased ventricular fibrosis, increased ejection fraction, and decreased LV end systolic diameter. To determine whether a natriuretic polypeptide is capable of inhibiting or reducing a symptom of acute HF, one or more of these parameters can be evaluated (e.g., before and after treatment with the natriuretic polypeptide), using methods known in the art, for example.

Variant natriuretic polypeptides having conservative and/or non-conservative substitutions (e.g., with respect to SEQ ID NO:3), as well as fragments of SEQ ID NO:3, fragments of variants of SEQ ID NO:3, and polypeptides comprising any of SEQ ID NO:3, variants or fragments of SEQ ID NO:3, or fragments of variants of SEQ ID NO:3, can be screened for biological activity using any of a number of assays, including those described herein. For example, the activity of a natriuretic polypeptide as described herein can be evaluated in vitro by testing its effect on cGMP production in cultured cells (e.g., cultured cardiac fibroblasts, aortic endothelial cells, or glomerular cells). Cells can be exposed to a natriuretic polypeptide (e.g., 10⁻⁹ to 10⁻⁴ M natriuretic polypeptide), and samples can be assayed to evaluate the natriuretic polypeptide's effects on cGMP generation. cGMP generation can be detected and measured using, for example, a competitive RIA cGMP kit (Perkin-Elmer, Boston, Mass.).

The activity of a natriuretic polypeptide also can be evaluated in vivo by, for example, testing its effects on factors such as plasma cGMP levels, urinary cGMP excretion, net renal generation of cGMP, glomerular filtration rate, blood pressure, heart rate, hemodynamic function such as cardiac output, pulmonary wedge pressure, systemic vascular resistance, and renal function such as renal blood flow, urine volume, and sodium excretion rate. In some cases, such parameters can be evaluated after inducing heart failure (e.g., by rapid right ventricular pacing).

### Nucleic Acids, Vectors, and Host Cells

This document also describes exemplary nucleic acids encoding polypeptides (e.g., natriuretic polypeptides), as well as expression vectors containing the nucleic acids, and host cells containing the nucleic acids and/or expression vectors. As used herein, the term “nucleic acid” refers to both RNA and DNA, including cDNA, genomic DNA, and synthetic (e.g., chemically synthesized) DNA. A nucleic acid molecule can be double-stranded or single-stranded (i.e., a sense or an antisense single strand). Nucleic acids include, for example, cDNAs encoding the natriuretic polypeptides and variant natriuretic polypeptides provided herein.

An “isolated nucleic acid” is a nucleic acid that is separated from other nucleic acid molecules that are present in a vertebrate genome, including nucleic acids that normally flank one or both sides of the nucleic acid in a vertebrate genome. The term “isolated” as used herein with respect to nucleic acids also includes any non-naturally-occurring nucleic acid sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences as well as DNA that is incorporated into a vector, an autonomously replicating plasmid, a virus (e.g., a retrovirus, lentivirus, adenovirus, or herpes virus), or into the genomic DNA of a prokaryote or eukaryote. In addition, an isolated nucleic acid can include an engineered nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not considered an isolated nucleic acid. By way of example and not limitation, an “isolated proANP nucleic acid,” for example, can be a RNA or DNA molecule containing 315 or more (e.g., 324 or more, 333 or more, 342 or more, 351 or more, 360 or more, 369 or more, or 378 or more) sequential nucleotide bases that encode at least a portion of proANP, or a RNA or DNA complementary thereto.

Also provided herein are nucleic acid molecules that can selectively hybridize under stringent hybridization conditions to a nucleic acid molecule encoding a natriuretic polypeptide (e.g., a nucleic acid molecule encoding a polypeptide having the amino acid sequence set forth in SEQ ID NO:3), or variants and fragments thereof. The term “selectively hybridize” means to detectably and specifically bind under hybridization and wash conditions that minimize appreciable amounts of detectable binding to nonspecific nucleic acids. For example, highly stringent or moderately stringent conditions can be used to achieve selective hybridization. Moderate and stringent hybridization conditions include those that are well known in the art. See, for example, sections 9.47-9.51 of Sambrook et al. (1989). For the purpose of this document, moderately stringent hybridization conditions mean the hybridization is performed at about 42° C. in a hybridization solution containing 25 mM KPO₄ (pH 7.4), 5×SSC, 5×Denhardt's solution, 50 μg/mL denatured, sonicated salmon sperm DNA, 50% formamide, 10% Dextran sulfate, and 1-15 ng/mL probe (about 5×10⁷ cpm/μg), while the washes are performed at about 50° C. with a wash solution containing 2×SSC and 0.1% sodium dodecyl sulfate. Highly stringent hybridization conditions mean the hybridization is performed at about 42° C. in a hybridization solution containing 25 mM KPO₄ (pH 7.4), 5×SSC, 5×Denhardt's solution, 50 μg/mL denatured, sonicated salmon sperm DNA, 50% formamide, 10% Dextran sulfate, and 1-15 ng/mL probe (about 5×10⁷ cpm/μg), while the washes are performed at about 65° C. with a wash solution containing 0.2×SSC and 0.1% sodium dodecyl sulfate.

Isolated nucleic acid molecules can be produced using standard techniques, including, without limitation, common molecular cloning and chemical nucleic acid synthesis techniques. For example, polymerase chain reaction (PCR) techniques can be used to obtain an isolated nucleic acid containing nucleotide sequence that encodes a natriuretic polypeptide as provided herein. PCR refers to a procedure or technique in which target nucleic acids are enzymatically amplified. Sequence information from the ends of the region of interest or beyond typically is employed to design oligonucleotide primers that are identical in sequence to opposite strands of the template to be amplified. PCR can be used to amplify specific sequences from DNA as well as RNA, including sequences from total genomic DNA or total cellular RNA. Primers typically are 14 to 40 nucleotides in length, but can range from 10 nucleotides to hundreds of nucleotides in length. General PCR techniques are described, for example in PCR Primer: A Laboratory Manual, ed. by Dieffenbach and Dveksler, Cold Spring Harbor Laboratory Press, 1995. When using RNA as a source of template, reverse transcriptase can be used to synthesize complementary DNA (cDNA) strands. Ligase chain reaction, strand displacement amplification, self-sustained sequence replication, or nucleic acid sequence-based amplification also can be used to obtain isolated nucleic acids. See, for example, Lewis (1992) Genetic Engineering News 12:1; Guatelli et al. (1990) Proc. Natl. Acad. Sci. USA 87:1874-1878; and Weiss (1991) Science 254:1292.

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector.

Isolated nucleic acids (e.g., nucleic acids encoding variant natriuretic polypeptides) also can be obtained by mutagenesis. For example, a reference sequence can be mutated using standard techniques including oligonucleotide-directed mutagenesis and site-directed mutagenesis through PCR. See, Short Protocols in Molecular Biology, Chapter 8, Green Publishing Associates and John Wiley & Sons, edited by Ausubel et al., 1992. Non-limiting examples of variant natriuretic polypeptides are provided herein.

This document also contemplates nucleic acid molecules encoding amino acid sequences from natriuretic polypeptides other than ANP, BNP, CNP, DNP, URO, or chimeras or variants thereof. Sources of nucleotide sequences from which nucleic acid molecules encoding a natriuretic polypeptide, or the nucleic acid complement thereof, can be obtained include total or polyA+ RNA from any eukaryotic source, including reptilian (e.g., snake) or mammalian (e.g., human, rat, mouse, canine, bovine, equine, ovine, caprine, or feline) cellular source from which cDNAs can be derived by methods known in the art. Other sources of the nucleic acid molecules provided herein include genomic libraries derived from any eukaryotic cellular source, including mammalian sources as exemplified above.

Nucleic acid molecules encoding native natriuretic polypeptides can be identified and isolated using standard methods, e.g., as described by Sambrook et al., Molecular Cloning: A Laboratory Manual, Cold Spring Harbor Laboratory Press, NY (1989). For example, reverse-transcriptase PCR (RT-PCR) can be used to isolate and clone natriuretic polypeptide cDNAs from isolated RNA that contains RNA sequences of interest (e.g., total RNA isolated from human tissue). Other approaches to identify, isolate and clone natriuretic polypeptide cDNAs include, for example, screening cDNA libraries.

Vectors containing nucleic acids such as those described herein also are provided. A “vector” is a replicon, such as a plasmid, phage, or cosmid, into which another DNA segment may be inserted so as to bring about the replication of the inserted segment. An “expression vector” is a vector that includes one or more expression control sequences, and an “expression control sequence” is a DNA sequence that controls and regulates the transcription and/or translation of another DNA sequence.

In the expression vectors provided herein, a nucleic acid (e.g., a nucleic acid encoding a natriuretic polypeptide) can be operably linked to one or more expression control sequences. As used herein, “operably linked” means incorporated into a genetic construct so that expression control sequences effectively control expression of a coding sequence of interest. Examples of expression control sequences include promoters, enhancers, and transcription terminating regions. A promoter is an expression control sequence composed of a region of a DNA molecule, typically within 100 to 500 nucleotides upstream of the point at which transcription starts (generally near the initiation site for RNA polymerase II). To bring a coding sequence under the control of a promoter, it is necessary to position the translation initiation site of the translational reading frame of the polypeptide between one and about fifty nucleotides downstream of the promoter. Enhancers provide expression specificity in terms of time, location, and level. Unlike promoters, enhancers can function when located at various distances from the transcription site. An enhancer also can be located downstream from the transcription initiation site. A coding sequence is “operably linked” and “under the control” of expression control sequences in a cell when RNA polymerase is able to transcribe the coding sequence into mRNA, which then can be translated into the protein encoded by the coding sequence. Expression vectors thus can be useful to produce antibodies as well as other multivalent molecules.

Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

An expression vector can include a tag sequence designed to facilitate subsequent manipulation of the expressed nucleic acid sequence (e.g., purification or localization). Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide including at either the carboxyl or amino terminus.

Host cells containing vectors also are provided. The term “host cell” is intended to include prokaryotic and eukaryotic cells into which a recombinant expression vector can be introduced. As used herein, “transformed” and “transfected” encompass the introduction of a nucleic acid molecule (e.g., a vector) into a cell by one of a number of techniques. Although not limited to a particular technique, a number of these techniques are well established within the art. Suitable methods for transforming and transfecting host cells can be found, for example, in Sambrook et al., Molecular Cloning: A Laboratory Manual (2^{nd} edition), Cold Spring Harbor Laboratory, New York (1989). For example, calcium phosphate precipitation, electroporation, heat shock, lipofection, microinjection, and viral-mediated nucleic acid transfer can be used introduce nucleic acid into cells. In addition, naked DNA can be delivered directly to cells in vivo as described elsewhere (U.S. Pat. Nos. 5,580,859 and 5,589,466).

### Compositions

The natriuretic polypeptides described herein (e.g., native natriuretic polypeptides, as well as variant natriuretic polypeptides), or nucleic acids encoding the natriuretic polypeptides described herein, can be incorporated into compositions for administration to a subject (e.g., a subject suffering from or at risk for cardiorenal disease). Methods for formulating and subsequently administering therapeutic compositions are well known to those in the art. Dosages typically are dependent on the responsiveness of the subject to the compound, with the course of treatment lasting from several days to several months, or until a suitable response is achieved. Persons of ordinary skill in the art routinely determine optimum dosages, dosing methodologies and repetition rates. Optimum dosages can vary depending on the relative potency of an antibody, and generally can be estimated based on the EC₅₀ found to be effective in in vitro and/or in vivo animal models. Compositions containing the compounds (e.g., natriuretic polypeptides) and nucleic acids provided herein may be given once or more daily, weekly, monthly, or even less often, or can be administered continuously for a period of time (e.g., hours, days, or weeks). For example, a natriuretic polypeptide or a composition containing a natriuretic polypeptide can be administered to a patient at a dose of at least about 0.01 ng natriuretic polypeptide/kg to about 100 mg natriuretic polypeptide/kg of body mass at or about the time of reperfusion, or can be administered continuously as an infusion beginning at or about the time of reperfusion and continuing for one to seven days (e.g., at a dose of about 0.01 ng natriuretic polypeptide/kg/minute to about 0.5 μg natriuretic polypeptide/kg/minute).

The natriuretic polypeptides and nucleic acids can be admixed, encapsulated, conjugated or otherwise associated with other molecules, molecular structures, or mixtures of compounds such as, for example, liposomes, receptor or cell targeted molecules, or oral, topical or other formulations for assisting in uptake, distribution and/or absorption.

In some embodiments, a composition can contain a natriuretic polypeptide as provided herein in combination with a pharmaceutically acceptable carrier. Pharmaceutically acceptable carriers include, for example, pharmaceutically acceptable solvents, suspending agents, or any other pharmacologically inert vehicles for delivering antibodies to a subject. Pharmaceutically acceptable carriers can be liquid or solid, and can be selected with the planned manner of administration in mind so as to provide for the desired bulk, consistency, and other pertinent transport and chemical properties, when combined with one or more therapeutic compounds and any other components of a given pharmaceutical composition. Typical pharmaceutically acceptable carriers include, without limitation: water; saline solution; binding agents (e.g., polyvinylpyrrolidone or hydroxypropyl methylcellulose); fillers (e.g., lactose or dextrose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

Pharmaceutical compositions containing molecules described herein can be administered by a number of methods, depending upon whether local or systemic treatment is desired. Administration can be, for example, parenteral (e.g., by subcutaneous, intrathecal, intraventricular, intramuscular, or intraperitoneal injection, or by intravenous (i.v.) drip); oral; topical (e.g., transdermal, sublingual, ophthalmic, or intranasal); or pulmonary (e.g., by inhalation or insufflation of powders or aerosols), or can occur by a combination of such methods. Administration can be rapid (e.g., by injection) or can occur over a period of time (e.g., by slow infusion or administration of slow release formulations).

Compositions and formulations for parenteral, intrathecal or intraventricular administration include sterile aqueous solutions (e.g., sterile physiological saline), which also can contain buffers, diluents and other suitable additives (e.g., penetration enhancers, carrier compounds and other pharmaceutically acceptable carriers).

Compositions and formulations for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders.

Formulations for topical administration include, for example, sterile and non-sterile aqueous solutions, non-aqueous solutions in common solvents such as alcohols, or solutions in liquid or solid oil bases. Such solutions also can contain buffers, diluents and other suitable additives. Pharmaceutical compositions and formulations for topical administration can include transdermal patches, ointments, lotions, creams, gels, drops, suppositories, sprays, liquids, and powders. Conventional pharmaceutical carriers, aqueous, powder or oily bases, thickeners and the like may be useful. In some embodiments, transdermal delivery of natriuretic polypeptides as provided herein can be particularly useful. Methods and compositions for transdermal delivery include those described in the art (e.g., in Wermeling et al. (2008) Proc. Natl. Acad. Sci. USA 105:2058-2063; Goebel and Neubert (2008) Skin Pharmacol. Physiol. 21:3-9; Banga (2007) Pharm. Res. 24:1357-1359; Malik et al. (2007) Curr. Drug Deliv. 4:141-151; and Prausnitz (2006) Nat. Biotechnol. 24:416-417).

Nasal preparations can be presented in a liquid form or as a dry product. Nebulized aqueous suspensions or solutions can include carriers or excipients to adjust pH and/or tonicity.

Pharmaceutical compositions include, but are not limited to, solutions, emulsions, aqueous suspensions, and liposome-containing formulations. These compositions can be generated from a variety of components that include, for example, preformed liquids, self-emulsifying solids and self-emulsifying semisolids. Emulsion formulations are particularly useful for oral delivery of therapeutic compositions due to their ease of formulation and efficacy of solubilization, absorption, and bioavailability. Liposomes can be particularly useful due to their specificity and the duration of action they offer from the standpoint of drug delivery.

Compositions provided herein can contain any pharmaceutically acceptable salts, esters, or salts of such esters, or any other compound which, upon administration to a subject, is capable of providing (directly or indirectly) the biologically active metabolite or residue thereof for the relevant compound (e.g., natriuretic polypeptide). Accordingly, for example, this document describes pharmaceutically acceptable salts of natriuretic polypeptides, prodrugs and pharmaceutically acceptable salts of such prodrugs, and other bioequivalents. A prodrug is a therapeutic agent that is prepared in an inactive form and is converted to an active form (i.e., drug) within the body or cells thereof by the action of endogenous enzymes or other chemicals and/or conditions. The term “pharmaceutically acceptable salts” refers to physiologically and pharmaceutically acceptable salts of the natriuretic polypeptides useful in methods provided herein (i.e., salts that retain the desired biological activity of the parent natriuretic polypeptides without imparting undesired toxicological effects). Examples of pharmaceutically acceptable salts include, but are not limited to, salts formed with cations (e.g., sodium, potassium, calcium, or polyamines such as spermine); acid addition salts formed with inorganic acids (e.g., hydrochloric acid, hydrobromic acid, sulfuric acid, phosphoric acid, or nitric acid); salts formed with organic acids (e.g., acetic acid, citric acid, oxalic acid, palmitic acid, or fumaric acid); and salts formed with elemental anions (e.g., bromine, iodine, or chlorine).

Compositions additionally can contain other adjunct components conventionally found in pharmaceutical compositions. Thus, the compositions also can include compatible, pharmaceutically active materials such as, for example, antipruritics, astringents, local anesthetics or anti-inflammatory agents, or additional materials useful in physically formulating various dosage forms of the compositions, such as dyes, flavoring agents, preservatives, antioxidants, opacifiers, thickening agents, and stabilizers. Furthermore, the composition can be mixed with auxiliary agents, e.g., lubricants, preservatives, stabilizers, wetting agents, emulsifiers, salts for influencing osmotic pressure, buffers, colorings, flavorings, penetration enhancers, and aromatic substances. When added, however, such materials should not unduly interfere with the biological activities of the other components within the compositions.

In some cases, a polypeptide provided herein can be formulated as a sustained release dosage form. For example, a natriuretic polypeptide can be formulated into a controlled release formulation. In some cases, coatings, envelopes, or protective matrices can be formulated to contain one or more of the polypeptides provided herein. Such coatings, envelopes, and protective matrices can be used to coat indwelling devices such as stents, catheters, and peritoneal dialysis tubing. In some cases, a polypeptide provided herein can incorporated into a polymeric substances, liposomes, microemulsions, microparticles, nanoparticles, or waxes.

Pharmaceutical formulations as disclosed herein, which can be presented conveniently in unit dosage form, can be prepared according to conventional techniques well known in the pharmaceutical industry. Such techniques include the step of bringing into association the active ingredients (i.e., the antibodies) with the desired pharmaceutical carrier(s). Typically, the formulations can be prepared by uniformly and intimately bringing the active ingredients into association with liquid carriers or finely divided solid carriers or both, and then, if necessary, shaping the product. Formulations can be sterilized if desired, provided that the method of sterilization does not interfere with the effectiveness of the molecules(s) contained in the formulation.

### Methods for Treating Cardiorenal Disease

This document also provides methods for using natriuretic polypeptides as disclosed herein for treating cardiorenal disease and components thereof. In some embodiments, for example, the compounds and nucleic acid molecules described herein can be administered to a mammal (e.g., a human or a non-human mammal) to treat ADHF. The composition or natriuretic polypeptide can be administered at any suitable dose, depending on various factors including, without limitation, the agent chosen and the patient characteristics. Administration can be local or systemic.

In some embodiments, a natriuretic polypeptide or a composition containing a natriuretic polypeptide can be administered at a dose of at least about 0.01 ng natriuretic polypeptide/kg to about 100 mg natriuretic polypeptide/kg of body mass (e.g., about 10 ng natriuretic polypeptide/kg to about 50 mg natriuretic polypeptide/kg, about 20 ng natriuretic polypeptide/kg to about 10 mg natriuretic polypeptide/kg, about 0.1 ng natriuretic polypeptide/kg to about 20 ng natriuretic polypeptide/kg, about 3 ng natriuretic polypeptide/kg to about 10 ng natriuretic polypeptide/kg, or about 50 ng natriuretic polypeptide/kg to about 100 μg/kg) of body mass, although other dosages also may provide beneficial results. A composition can be administered at a dose of, for example, about 0.1 ng natriuretic polypeptide/kg/minute to about 500 ng natriuretic polypeptide/kg/minute (e.g., about 0.5 ng natriuretic polypeptide/kg/minute, about 1 ng natriuretic polypeptide/kg/minute, about 2 ng natriuretic polypeptide/kg/minute, about 3 ng natriuretic polypeptide/kg/minute, about 5 ng natriuretic polypeptide/kg/minute, about 7.5 ng natriuretic polypeptide/kg/minute, about 10 ng natriuretic polypeptide/kg/minute, about 12.5 ng natriuretic polypeptide/kg/minute, about 15 ng natriuretic polypeptide/kg/minute, about 20 ng natriuretic polypeptide/kg/minute, about 25 ng natriuretic polypeptide/kg/minute, about 30 ng natriuretic polypeptide/kg/minute, about 50 ng natriuretic polypeptide/kg/minute, about 100 ng natriuretic polypeptide/kg/minute, or about 300 ng natriuretic polypeptide/kg/minute).

In some embodiments, a natriuretic polypeptide or a composition containing a natriuretic polypeptide can be administered via a first route (e.g., intravenously) for a first period of time, and then can be administered via another route (e.g., topically or subcutaneously) for a second period of time. For example, a composition containing a natriuretic polypeptide can be intravenously administered to a mammal (e.g., a human) at a dose of about 0.1 ng natriuretic polypeptide/kg/minute to about 300 ng natriuretic polypeptide/kg/minute (e.g., about 1 ng natriuretic polypeptide/kg/minute to about 15 ng natriuretic polypeptide/kg/minute, about 3 ng natriuretic polypeptide/kg/minute to about 10 ng natriuretic polypeptide/kg/minute, or about 10 ng natriuretic polypeptide/kg/minute to about 30 ng natriuretic polypeptide/kg/minute) for one to seven days (e.g., one, two, three, four, five, six, or seven days), and subsequently can be subcutaneously administered to the mammal at a dose of about 10 ng natriuretic polypeptide/kg/day to about 100 ng natriuretic polypeptide/kg/day (e.g., about 10 ng natriuretic polypeptide/kg/day, about 20 ng natriuretic polypeptide/kg/day, about 25 ng natriuretic polypeptide/kg/day, about 30 ng natriuretic polypeptide/kg/day, about 50 ng natriuretic polypeptide/kg/day, or about 100 ng natriuretic polypeptide/kg/day) for five to 30 days (e.g., seven, 10, 14, 18, 21, 24, or 27 days).

The methods provided herein can include administering to a mammal an effective amount of a natriuretic polypeptide (e.g., a native or variant natriuretic polypeptide) or a nucleic acid encoding a natriuretic polypeptide, or an effective amount of a composition containing such a molecule. As used herein, the term “effective amount” is an amount of a molecule or composition that is sufficient to alter the desired parameter by at least 10%. For example, in some embodiments, an “effective amount” of a natriuretic polypeptide can be an amount of the natriuretic polypeptide that is sufficient to increase natriuresis and/or diuresis (or a characteristic of natriuresis and/or diuresis such as plasma cGMP levels, urinary cGMP excretion, net renal cGMP generation, urine flow, urinary sodium excretion, urinary potassium excretion, hematocrit, plasma BNP immunoreactivity, renal blood flow, plasma ANP immunoreactivity, renal vascular resistance, proximal and distal fractional reabsorption of sodium, mean arterial pressure, pulmonary capillary wedge pressure, right atrial pressure, pulmonary arterial pressure, plasma renin activity, plasma angiotensin II levels, plasma aldosterone levels, renal perfusion pressure, and systemic vascular resistance) by at least 10% (e.g., 10%, 15%, 20%, 25%, 30%, 40%, 50%, 60%, 70%, 75%, 80%, 85%, 90%, 95%, 99%, or 100%), as compared to the level of the same parameter prior to treatment, or as compared to the level of the parameter in a control, untreated mammal. For example, an “effective amount” of a natriuretic polypeptide can be an amount that increases sodium excretion in a treated mammal by at least 10% as compared to the level of sodium excretion in the mammal prior to administration of the natriuretic polypeptide, or as compared to the level of sodium excretion in a control, untreated mammal.

In some embodiments, an “effective amount” of a natriuretic polypeptide can be an amount of the natriuretic polypeptide that is sufficient to reduce the occurrence of a symptom of cardiorenal disease by at least 10% (e.g., 10%, 15%, 20%, 25%, 30%, 40%, 50%, 60%, 70%, 75%, 80%, 85%, 90%, 95%, 99%, or 100%). In some cases, for example, an “effective amount” of a natriuretic polypeptide as provided herein can be an amount that reduces a symptom of ADHF in a treated mammal by at least 10% as compared to the level of the symptom in the mammal prior to administration of the natriuretic polypeptide or without administration of the natriuretic polypeptide, or as compared to the level of the symptom in a control, untreated mammal. The presence or extent of such symptoms can be evaluated using methods known in the art.

Before administering a composition provided herein to a mammal, the mammal can be assessed to determine whether or not the mammal has a need for treatment for cardiorenal disease. After identifying a mammal as being in need of treatment, the mammal can be treated with a composition provided herein. For example, a composition containing one or more polypeptides having a natriuretic polypeptide activity (e.g., a proANP polypeptide) can be administered to a mammal in any amount, at any frequency, and for any duration effective to achieve a desired outcome (e.g., to reduce atrial fibrillation).

In some embodiments, the amount and frequency of natriuretic polypeptide administered to a mammal can be titrated in order to, for example, identify a dosage that is most effective to treat cardiorenal disease while having the least amount of adverse effects. For example, an effective amount of a composition can be any amount that reduces fibrillation within a mammal without having significant toxicity in the mammal. If a particular mammal fails to respond to a particular amount, then the amount can be increased by, for example, tenfold. After receiving this higher concentration, the mammal can be monitored for both responsiveness to the treatment and toxicity symptoms, and adjustments in the dosage can be made accordingly. The effective amount can remain constant or can be adjusted as a sliding scale or variable dose depending on the mammal's response to treatment.

The frequency of administration can be any frequency that reduces a symptom of cardiorenal disease within a mammal without producing significant toxicity in the mammal. For example, the frequency of administration can be from about four times a day to about once every other month, or from about once a day to about once a month, or from about once every other day to about once a week. In addition, the frequency of administration can remain constant or can be variable during the duration of treatment. As with the effective amount, various factors can influence the actual frequency of administration used for a particular application. For example, the effective amount, duration of treatment, route of administration, and severity of renal condition may require an increase or decrease in administration frequency.

An effective duration of administration can be any duration that reduces a symptom of cardiorenal disease within a mammal without producing significant toxicity in the mammal. The effective duration can vary from several days to several weeks, months, or years. In general, the effective duration for can range in duration from several days to several months. For example, an effective duration can range from about one to two weeks to about 36 months. Prophylactic treatments can be typically longer in duration and can last throughout an individual mammal's lifetime. Multiple factors can influence the actual effective duration used for a particular treatment or prevention regimen. For example, an effective duration can vary with the frequency of administration, amount administered, route of administration, and severity of a renal condition.

After administering a composition provided herein to a mammal, the mammal can be monitored to determine whether the cardiorenal disease has improved. For example, a mammal can be assessed after treatment to determine whether or not ADHF has been reduced. As described herein, any method can be used to assess improvements in ADHF.

The invention will be further described in the following examples, which do not limit the scope of the invention described in the claims.

### EXAMPLES

Experiments were conducted to assess the processing and degradation of proANP in the circulation and in the kidney as compared to the processing and degradation of ANP.

### Example 1

### Materials and Methods

Animals, Groups, Surgical Procedure, and Samplings:

Studies were conducted in four groups (n=5 for each) of male mongrel dogs (weight, 20 to 28 kg) in normal and ADHF canines Groups included: Group 1 (A/B): proANP injection; and Group 2 (A/B): ANP injection (A: normal and B: ADHF). Heart failure was produced by rapid right ventricular pacing (240 bpm) for 10 days as reported elsewhere (Ichiki et al., Am J Physiol Regul Integr Comp Physiol, 2013, 304:R102-R109 (epub 2012, doi:10.1152/ajpregu.00233); Chen et al., J Am Coll Cardiol, 2009, 53:1302-1308; Boerrigter et al., Hypertension, 2007, 49:1128-1133; and Costello-Boerrigter et al., Circ Heart Fail, 2010, 3:412-419). Blood samples were obtained from aorta and renal vein and urine is obtained before and 2.5, 5, 10, 15, 30, 60, 120, and 180 min after injection. Canines were then euthanized for tissue harvest, and kidney medulla and cortex were collected for analysis as well. Dogs were injected with equimolar doses of canine proANP or ANP (667 pmol/kg) based on preliminary data and a study published elsewhere (Nishida et al. Jpn J Physiol, 1990, 40:531-540). These doses were similar to doses of carperitide/nesiritide in clinical use.

Mass Spectrometry Immunoassay:

Protease inhibition and quantitative mass spectrometry immunoassay (MSIA, Intrinsic Bioprobes, Hayward, Calif.) were used, as described elsewhere (Miller et al., Int J Clin Chem, 2012; Hawkridge et al., Proc Natl Acad Sci USA, 2005, 102:17442-17447; and Niederkofler et al., Circulation. Heart Failure, 2008, 1:258-264). In brief, MSIA protein extraction was achieved using antibody derivatized affinity pipettes (Intrinsic Bioprobes, Inc.). These affinity pipettes or MSIA-Tips also were produced as reported elsewhere (Niederkofler et al., supra). A mixture of equal concentrations (45 ug/ml) of Mabs 8.1 and 106.3 was used as the affinity ligand. For nonspecific MSIA affinity tips, an unrelated antibody against antihuman beta2-microglobulin (Dako-Cytomation) was coupled. Plasma was thawed at room temperature (RA) and incubated at 37° C. for 10 minutes. Warmed plasma was centrifuged at 5000 g, and 500 ul aliquots were transferred into a 96-well sample tray. Biotinylated ANP 1-28 or URO was added to each well at a final concentration of 500 pg/ml to serve as an internal reference for MSIA. Samples were diluted 2-fold with HBS-EP containing 1% Tween20 and 1 mol/1 NaC1 before MSIA. Analyses of spiked standard curve samples, HF samples, and healthy control samples were performed in parallel. Samples (1 ml) were first drawn 50 times through beta2-microglobulin affinity tips by automated aspiration/dispensing of 150 ul, followed by 300 times through ANP/URO affinity tips. Next, each affinity tip was rinsed sequentially with HBS-EP, water, 25% acetonitrile in 2 mol/l ammonium acetate, 50 mmol/l n-octyl glucoside, and water (15 aspirations/dispenses at 150 ul for each step). Bound proteins was eluted from the affinity tip with 3.5 ul of matrix assisted laser desorption ionization (MALDI) matrix solution and deposited directly onto the MALDI-target. Mass spectra were acquired for each eluent by summing six 250-laser shot acquisitions using a linear Bruker Autoflex MALDI-time of flight (TOF) mass spectrometer. Resulting mass spectra were batch processed in Flex Analysis 2.4 (Bruker Daltonics) to label all spectral peaks with a signal-to-noise >20 and to internally calibrate the m/z axis using bANP. In addition, spectral intensities were normalized to the peak intensities of ANP for intersample comparison and quantification. Peak lists containing peak characteristics were imported into spreadsheets for data analysis. For analysis of renal tissue, kidney tissue was minced and homogenates were rapidly prepared on ice. Homogenates were quickly frozen for future mass spec analysis as done for plasma and urine. A Physician-Scientist with expertise in natriuretic peptide biomarker measurements and in mass spec analysis oversaw and assisted with quality control and troubleshooting on mass spec.

Cyclic GMP Assay:

The samples were ether extracted four times in 4 volumes of ether, dried, and reconstituted in 300 ul cGMP assay buffer, and the cGMP levels were measured using a competitive RIA cGMP kit (Perkin-Elmer, Boston, Mass.).

Other Assays:

Canine ANP1-28 and NT-proANP concentrations are determined by specific immunoassays as described elsewhere (Ichiki et al., Clin Chem, 2011, 57:40-47; Ichiki et al., 2012, supra; Chen et al., J Am Coll Cardiol, 2000, 35:240A; and Chen et al., Am J Physiol Regul Integr Comp Physiol, 2005, 288:R1093-1097).

Statistical Analysis:

Continuous variables are expressed as mean±SEM. Normal distributed data is assessed by Student's unpaired t-tests between groups, followed by Fisher's least significant difference test when appropriate. For non-nominal distributed data, Wisconsin rank sum test is performed. Nominal distribution of respective data is determined by the Shapiro-Wilk test. Statistical significance is accepted at p<0.05. As this is highly technical and labor intensive, a power calculation is not used. Rather, an N of 5 in each group is used, based in part on initial mass spec reports on molecular forms of BNP human plasma (Hawkridge et al., supra).

### Example 2

### ProANP is Processed and Degraded in Human Serum Ex Vivo

Experiments were conducted to investigate whether exogenous proANP is processed into its active form in human circulation. proANP tagged with histidine at the C-terminus (FIG. 4A) was incubated in human serum, and his-tag protein was isolated and detected by his-tag antibody as described elsewhere (Ichiki et al., 2011, supra). In normal serum, proANP was processed into ANP1-28 after a 5 minute incubation, and the amount of ANP1-28 decreased in 180 minutes, consistent with degradation (FIG. 4B). Compared to normals, proANP processing into ANP1-28 was preserved in HF (FIG. 4C), suggesting that proANP is processed in both normal and HF circulation and may have a longer half-life than ANP.

### Example 3

### ProANP and its Processed Form Stimulate cGMP Production in HEK293 Cells and Human Cardiac Fibroblasts In Vitro

To assess whether proANP is an active peptide in vitro compared with mature ANP or BNP, receptor activating activity was examined based on second messenger cGMP production in renally derived human kidney (HEK293) cells overexpressing GC-A or GC-B. Mature NPs, ANP1-28, and BNP1-32 stimulated cGMP production in cells overexpressing GC-A, but not in cells overexpressing GC-B. proANP also stimulated GC-A-overexpressing cells to an extent that was lower than ANP but much higher than proBNP (FIG. 5A). Processed proANP treated in serum for 5 minutes stimulated cGMP production significantly as compared to untreated proANP, but stimulation of cGMP production by proANP treated in serum for 180 minutes was decreased (FIG. 5B).

Further studies utilized human CFs in which GC-A and proANP convertase corin exist (FIG. 5C). ProANP stimulated cGMP production to a level that was about half as much as ANP for the first 30 minutes, but after 60 minutes stimulation by proANP and ANP was about equal (FIG. 5D). These results suggested that proANP itself is an active peptide that may have more prolonged effects than mature NPs.

### Example 4

### Renal Specific Action of proANP as Compared to ANP1-28 and BNP1-32 in Normal Canines

The in vivo effects of canine proANP, ANP1-28, and BNP1-32 were examined in normal canines (FIG. 6). After an equimolar intravenous injection of peptides (667 pmol/kg, based on studies reported elsewhere; Nishida et al., supra) which is similar to the dose of carperitide/nesiritide used clinically, ANP and BNP immunoreactivity increased rapidly after the injection and peaked at 2 minutes, and then decreased rapidly (FIG. 6A). proANP also increased rapidly and peaked at 2 minutes, but decreased more slowly than ANP or BNP (FIG. 6A). Responses also were seen in cGMP immunoreactivity (FIG. 6B). In pharmacokinetic analysis (Table 1 and FIG. 7), proANP had a half-life that was about 4 times longer ANP, and its area under the curve (AUC) for NP immunoreactivity was twice that of ANP. Importantly, the AUC for urinary cGMP excretion (UcGMP) with proANP was twice that of ANP or BNP, whereas AUC for plasma cGMP (PcGMP) with proANP was less than that for ANP or BNP, suggesting proANP has longer half-life as a renal acting hormone. In regard to hemodynamic responses, BNP had the greatest effect on blood pressure (BP), while proANP had less of an effect on BP as compared to the other two NPs (FIG. 6C). The effect of proANP on heart rate was less than that of ANP or BNP for the first 90 minutes, but then increased by about 120 minutes (FIG. 6D). proANP's effect on cardiac output was less than that of either ANP or BNP (FIG. 6E). However, proANP had the strongest and longest diuretic and natriuretic actions of these three NPs (FIGS. 6F and 6G), while having less effect on renal blood flow (FIG. 6H). These studies indicate that proANP is a potent diuretic and natriuretic peptide, with minimal systemic hemodynamic actions.

TABLE 1

| | NPi Cmax | NPi AUC | Half-life | PcGMP Cmax | PcGMP AUC | UcGMP Cmax | UcGMP AUC |
|---|---|---|---|---|---|---|---|
| | (pmol/ml) | (pmol · min/ml) | (min) | (pmol/ml) | (pmol · min/ml) | (pmol/ml) | (pmol · min/ml) |
| proANP | 0.96 | 28.1 | 20.7 | 93.8 | 6110.5 | 7027.8 | 420368 |
| ANP | 0.81 | 12 | 5.1 | 133.6 | 7760.8 | 6820.9 | 237037 |
| BNP | 0.43 | 3.1 | 3.7 | 255.4 | 13406.9 | 6936.2 | 207454 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for treating a cardiorenal disease in a mammal in need thereof, comprising administering to said mammal a proANP polypeptide in an amount effective to reduce a symptom of said cardiorenal disease.

2. The method of claim 1, wherein said cardiorenal disease comprises acute decompensated heart failure.

3. The method of claim 1, wherein said proANP polypeptide comprises amino acids 21-126 of SEQ ID NO:3.

4. The method of claim 1, wherein said proANP polypeptide comprises SEQ ID NO:3.

5. The method of claim 1, wherein said proANP polypeptide consists of SEQ ID NO:3.

6. The method of claim 1, comprising administering to said mammal a composition comprising said proANP polypeptide.

7. The method of claim 1, wherein said mammal is a human.

8. The method of claim 1, wherein said proANP polypeptide is administered at a dose of 0.01 ng/kg to 50 ug/kg

9. The method of claim 1, comprising administering said proANP intravenously.

10. The method of claim 1, wherein said symptom is selected from the group consisting of as edema, shortness of breath, and fatigue, cardiac unloading, increased glomerular filtration rate, decreased plasma renin activity, decreased levels of angiotensin II, decreased proliferation of cardiac fibroblasts, decreased left ventricular (LV) hypertrophy, decreased LV mass, decreased pulmonary wedge capillary pressure, decreased right atrial pressure, decreased mean arterial pressure, decreased levels of aldosterone, decreased ventricular fibrosis, increased ejection fraction, and decreased LV end systolic diameter.

11. The method of claim 1, further comprising identifying said mammal as being in need of said treatment.


SEQUENCE LISTING (11 sequences)

SEQ ID NO: 1 - protein, 151 aa, Homo sapiens
MSSFSTTTVSFLLLLAFQLLGQTRANPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 2 - protein, 149 aa, Canis lupus familiaris
MGSPIAASFLLFLAVQLLGQTRANPVYGSVSNADLLDFKNLLDRLEDKMPLEDEAESPQALSEQNAEAGAALSPLPEVPPWTGEVSPAQRDGGALGRGPWDSSDRSALLKSKLRALLAAPRSLRRSSCFGGRMDRIGAQSGLGCNSFRY

SEQ ID NO: 3 - protein, 126 aa, Homo sapiens
NPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPRSLRRSSCFGGRMDRIGAQSGLGCNSFRY

SEQ ID NO: 4 - protein, 28 aa, Homo sapiens
SLRRSSCFGGRMDRIGAQSGLGCNSFRY

SEQ ID NO: 5 - protein, 98 aa, Homo sapiens
NPMYNAVSNADLMDFKNLLDHLEEKMPLEDEVVPPQVLSEPNEEAGAALSPLPEVPPWTGEVSPAQRDGGALGRGPWDSSDRSALLKSKLRALLTAPR

SEQ ID NO: 6 - protein, 32 aa, Homo sapiens
TAPRSLRRSSCFGGRMDRIGAQSGLGCNSFRY

SEQ ID NO: 7 - protein, 94 aa, Homo sapiens
NPMYNAVSNADLMDFKNLLDHLEEKMPLEDEVVPPQVLSEPNEEAGAALSPLPEVPPWTGEVSPAQRDGGALGRGPWDSSDRSALLKSKLRALL

SEQ ID NO: 8 - protein, 126 aa, Canis lupus familiaris
NPVYGSVSNADLLDFKNLLDRLEDKMPLEDEAESPQALSEQNAEAGAALSPLPEVPPWTGEVSPAQRDGGALGRGPWDSSDRSALLKSKLRALLAAPRSLRRSSCFGGRMDRIGAQSGLGCNSFRY

SEQ ID NO: 9 - protein, 32 aa, Homo sapiens
SPKMVQGSGCFGRKMDRISSSSGLGCKVLRRH

SEQ ID NO: 10 - protein, 22 aa, Homo sapiens
GLSKGCFGLKLDRIGSMSGLGC

SEQ ID NO: 11 - protein, 38 aa, Dendroaspis augusticeps
EVKYDPCFGHKIDRINHVSNLGCPSLRDPRPNAPSTSA